(19) United States
(12) Patent Application Publication
(10) Pub. No.: US 2010/0239661 A1
(43) Pub. Date: Sep. 23, 2010

(54) PHARMACEUTICAL COMPOSITIONS OF URSODIOL

(76) Inventors: Sunilendu Bhushan Roy, Gujarat (IN); Sushrut Krishnaji Kulkarni, Gujarat (IN); Babulal Patel Dharmesh, Gujarat (IN)

Correspondence Address: LADAS & PARRY LLP, 26 WEST 61ST STREET, NEW YORK, NY, 10023, US

(21) Appl. No.: 12/446,842

(22) PCT Filed: Oct. 23, 2007
(86) PCT No.: PCT/IN07/00504

§ 371 Date: Mar. 31, 2010

(30) Foreign Application Priority Data
Oct. 26, 2006 (IN) 1773/MUM/2006

(51) Int. Cl. A61K 9/48; A61K 9/14; A61K 31/575; A61K 9/20; A61P 1/00

(52) U.S. Cl. 424/452; 424/489; 514/182; 424/465

(57) ABSTRACT

A pharmaceutical composition is disclosed. The composition comprises a therapeutically effective amount of active pharmaceutical ingredient that is Ursodiol or its pharmaceutically acceptable salts thereof with one or more pharmaceutically acceptable excipients, wherein said active pharmaceutical ingredient present in the form of micronized and unmicronized particles in the ratio about 5:95 to about 95:5.

### FIELD OF INVENTION

The technical field of the invention relates to solid pharmaceutical compositions of Ursodiol with enhanced absorption and dissolution characteristics provided by micronizing the Ursodiol.

### BACKGROUND OF INVENTION

Ursodiol (ursodeoxycholic acid, UDCA) is a naturally occurring bile acid found in small quantities in normal human bile and in larger quantities in the biles of certain species of bears. It is a bitter-tasting white powder consisting of crystalline particles freely soluble in ethanol and glacial acetic acid, slightly soluble in chloroform, sparingly soluble in ether, and practically insoluble in water. The chemical name of ursodiol is 3α,7β-dihydroxy-5β-cholan-24-oic acid (C₂₄H₄₀O₄). Ursodiol has a molecular weight of 392.56. Its structure is shown below.

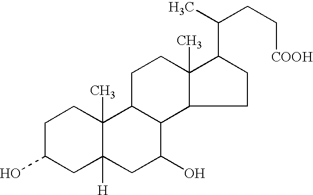

Ursodiol (UDCA) is normally present as a minor fraction of the total bile acids in humans (about 5%). Following oral administration, the majority of ursodiol is absorbed by passive diffusion and its absorption is incomplete. Once absorbed, ursodiol undergoes hepatic extraction to the extent of about 50% in the absence of liver disease. As the severity of liver disease increases, the extent of extraction decreases. In the liver, ursodiol is conjugated with glycine or taurine, then secreted into bile. These conjugates of ursodiol are absorbed in the small intestine by passive and active mechanisms. The conjugates can also be deconjugated in the ileum by intestinal enzymes, leading to the formation of free ursodiol that can be reabsorbed and reconjugated in the liver. Nonabsorbed ursodiol passes into the colon where it is mostly 7-dehydroxylated to lithocholic acid. Some ursodiol is epimerized to chenodiol (CDCA) via a 7-oxo intermediate. Chenodiol also undergoes 7-dehydroxylation to form lithocholic acid.

These metabolites are poorly soluble and excreted in the feces. A small portion of lithocholic acid is reabsorbed, conjugated in the liver with glycine, or taurine and sulfated at the 3 position. The resulting sulfated lithocholic acid conjugates are excreted in bile and then lost in feces.

Lithocholic acid, when administered chronically to animals, causes cholestatic liver injury that may lead to death from liver failure in certain species unable to form sulfate conjugates. Ursodiol is 7-dehydroxylated more slowly than chenodiol. For equimolar doses of ursodiol and chenodiol, steady state levels of lithocholic acid in biliary bile acids are lower during ursodiol administration than with chenodiol administration. Humans and chimpanzees can sulfate lithocholic acid. Although liver injury has not been associated with ursodiol therapy, a reduced capacity to sulfate may exist in some individuals. Nonetheless, such a deficiency has not yet been clearly demonstrated and must be extremely rare, given the several thousand patient-years of clinical experience with ursodiol.

In healthy subjects, at least 70% of ursodiol (unconjugated) is bound to plasma protein. No information is available on the binding of conjugated ursodiol to plasma protein in healthy subjects or primary biliary cirrhosis (PBC) patients. Its volume of distribution has not been determined, but is expected to be small since the drug is mostly distributed in the bile and small intestine. Ursodiol is excreted primarily in the feces. With treatment, urinary excretion increases, but remains less than 1% except in severe cholestatic liver disease.

During chronic administration of ursodiol, it becomes a major biliary and plasma bile acid. At a chronic dose of 13 to 15 mg/kg/day, ursodiol constitutes 30-50% of biliary and plasma bile acids.

Ursodiol available in market as URSO 250® URSO Forte™ and Actigall® brands.

There is an ever-present need in the pharmaceutical industry for improved pharmaceutical formulations that enhance the efficacy of poorly soluble therapeutic agents. There is especially a need for formulations that enhance the absorption of poorly soluble therapeutic agents.

The aqueous solubility of drug substances plays an important role in the formulation of dosage forms. For the oral route of administration it is well experienced that, unless the substance has an aqueous solubility above 10 mg/ml over the pH range 1-7, potential absorption problems may occur. Numerous active ingredients suffer from the disadvantage of being poorly soluble in an aqueous medium, thus having an insufficient dissolution profile and consequently, poor bioavailability following oral administration.

Ursodiol as a drug, which is characterized as having low solubility and high permeability. Oral absorption of Ursodiol is rapid and complete. Kinetic profile of Ursodiol shows that when in-vivo drug dissolution is complete, there is no constraint to absorption. Ursodiol has low solubility, and thus, the rate limiting step for oral bioavailability of Ursodiol is the dissolution of the drug from the pharmaceutical dosage form.

Accordingly, there remains a need for improved pharmaceutical formulations of Ursodiol, which improve the dissolution of Ursodiol, thereby increasing its bioavailability.

Surprisingly, the inventors have discovered that dosage form prepared using micronized and unmicronized Ursodiol, results in an improved dissolution and absorption characteristics.

### SUMMARY OF THE INVENTION

In accordance with one embodiment of the present invention, there is provided a pharmaceutical composition comprising a therapeutically effective amount of active pharmaceutical ingradient that is Ursodiol or its pharmaceutically acceptable salts thereof with one or more pharmaceutically acceptable excepients, wherein said active pharmaceutical ingradient present in the form of micronized and unmicronized particles in the ratio of about 5:95 to about 95:5 and preferably about 20:80 to about 80:20.

More preferably said micronized and unmicronized particles in pharmaceutical composition are present in ratio between about 30:70 to about 70:30. Most preferably said micronized and unmicronized particles in pharmaceutical composition are present in ratio between about 40:60 to about 60:40.

The micronized particles according to the present invention means Ursodiol having an average particle size (d90) not more than about 20 micron, preferably less than 10 microns, more preferably said micronized particles have an average particle size less than about 7 microns.

The unmicronized particles according to the present invention means Ursodiol having an average particle size (d90) not less than about 150 microns and preferably said unmicronized particles having an average particle size not more than about 350 microns.

The present invention provides a pharmaceutical composition comprising Ursodiol in the form of mixture of micronized and unmicronized particles.

The present invention further provide a process for preparing pharmaceutical composition of Ursodiol, which comprises micronizing Ursodiol to obtain micronized ursodiol having particle size d90 less than 20 micron, mixing micronzed ursodilol with unmicronized ursodiol and one or more pharmaceutically acceptable excepients.

The process of the invention for preparing a solid formulation of Ursodiol with improved dissolution and absorption characteristics includes the micronization of Ursodiol. Size reduction, or micronization, may be carried out using any of the conventionally known mills, such as a ball mill, colloid mill, grinding mill, air jet mill, roller mill, impact mill, etc. Air jet milling is particularly well suited for this application as it is a well proven technique that consistently produces particles of a size less than 35 microns, preferably less than 20 micron. Primary advantages of air jet milling are that the predominant particle size reduction occurs through particle to particle collisions, there is limited particle size reduction that results from metal to product contact, and there is no generation of heat that can adversely affect the particles being micronized.

The process of air jet milling involves exposing the material to be micronized to streams of compressed air or gas. Particles in the fluidized bed created by the gas streams are accelerated towards the center of the mill and collide with the slower moving particles. These collisions break the particles into smaller particles, thereby micronizing the particles. The air jet mills operate by applying opposing airflows and centrifugal forces. By balancing the two forces, desired particle size and fines can be separated.

In the preferred embodiment, Ursodiol is micronized to obtain reduced particle size e.g. d₉₀ particle size of less than 20 micron, preferably less than 10 micron, and more particularly less than 7 micron. Thus obtained micronized Ursodiol particles are used in combination with unmicronized particles of Ursodiol to provide an improved bioavailability of Ursodiol pharmaceutical compositions as compared to Ursodiol pharmaceutical compositions that contain only, normal particle size of Ursodiol. Ursodiol micronized particles having a d₉₀ particle size of less than about 10 microns, and more particularly less than about 7 microns. As used herein, “D₉₀ particle size” is the particle size of at least 90% of the particles of micronized Ursodiol.

Further the pharmaceutical composition comprising one or more pharmaceutically acceptable excipient(s).

Pharmaceutically acceptable excipient(s) includes disintegrants, diluents, binder, flavoring agents, lubricants, fillers, glidents, sweetening agents, coloring agents, stabilizers, masking agents, wetting agents, suspending agents and the like.

The pharmaceutical composition is in the form of tablet or capsule or a powder suspension in a liquid or aerosol, preferably in the form of tablet or capsule.

The pharmaceutical composition provides immediate or modified release of the ursodiol particles.

The term “therapeutically effective amount” as used herein means the amount of a′ compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated. As used herein, the term “binders” is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, polypropylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art. As used herein, the term “diluent” or “filler” is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

As used herein, the term “glidant” is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

As used herein, the term “lubricant” is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

As used herein, the term “disintegrant” is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

As used herein, the term “wetting agent” is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

The invention described here is demonstrated in examples illustrated below. These examples are provided as illustration only and therefore should not be construed as limitation of the scope of invention.

### EXAMPLE 1

### Ursodiol Tablet Formulation

| | | Example A | Example B |
|---|---|---|---|
| Sr. No. | Ingredients | mg/tab | mg/tab |
| 1 | Ursodiol | 350.00 | 300.00 |
| | (Micronized particle having D90 | | |
| | value less than 10 microns) | | |
| 2 | Ursodiol | 150.00 | 200.00 |
| | (Unmicronized particle having D90 | | |
| | value more than 170 microns) | | |
| 3 | Sodium Starch Glycolate | 8.00 | 8.00 |
| 4 | Microcrystalline Cellulose | 97.00 | 97.00 |
| | (Avicel PH 101) | | |
| 5 | Polyethylene Glycol 3350 | 24.00 | 24.00 |
| 6 | Povidone K 30 | 24.00 | 24.00 |
| 7 | Purified Water | q.s. | q.s |
| 8 | Syloid 244 FP | 12.00 | 12.00 |
| 9 | Sodium Starch Glycolate | 40.00 | 40.00 |
| 10 | Microcrystalline Cellulose | 87.00 | 87.00 |
| | (Avicel PH 102) | | |
| 11 | Magnesium Stearate | 8.00 | 8.00 |
| | Total (Core wt.) | 800.00 | 800.00 |
| 12 | Opadry white 03F58991 | 8.00 | 8.00 |
| 13 | Purified Water | 808.00 | 808.00 |

Ursodiol Tablets USP, 250 mg and 500 mg are dose proportional formula.

### Procedure

1. Ingredients 1, 2, and 3 were mixed in RMG for 10 minutes at impeller fast speed and chopper off.
2. Ingredients 4 and 5 were dissolved in sufficient qty. of Purified water.
3. Solution of step no. 2 was used as a binder solution and added in RMG within 2 minutes at impeller fast speed and chopper off.
4. Mix the above mixture for sufficient time at impeller fast and chopper fast speed.
5. If required, required qty. of purified water was added in RMG at above condition to get suitable granular mass.
6. Wet mass was transferred in FBD, dry the granules at 50° C. to 60° C. to achieve the LOD at 105° C. below 1.5% w/w.
7. Dry granules were passed through oscillating granulator fitted with 16# mesh along with Syloid 244 FP.
8. Granules were transferred in blender and mixed for 5 minutes.
9. Ingredients 8 and 9 were transferred in blender and mixed for 10 minutes.
10. Ingredient 10 was transferred in blender and mixed for 3 minutes.
11. Compress the above blend and coating was done with 1% w/w of core tablet weight of Opadry white 03F58991 of 10% w/w solution dispersion.

### Following is Dissolution Data for the Above Said Examples A and B.

### Dissolution Profile of Ursodiol Tablets USP, 500 mg of Example A.

| Medium | Simulated Intestinal Fluid pH 8.0 (USP media) |
|---|---|
| Volume | 900 ml |
| Apparatus | Paddle (USP II) |
| RPM | 75 |

| Time (min.) | % Dissolution |
|---|---|
| 5 | 67.8 |
| 10 | 89.9 |
| 15 | 95.8 |
| 30 | 96.4 |
| 45 | 96.7 |
| 60 | 97.5 |

### Dissolution Profile of Ursodiol Tablets USP, 500 mg of Example B.

| Medium | Simulated Intestinal Fluid pH 8.0 (USP media) |
|---|---|
| Volume | 900 ml |
| Apparatus | Paddle (USP II) |
| RPM | 75 |

| Time (min.) | % Dissolution |
|---|---|
| 5 | 69.30 |
| 10 | 94.00 |
| 15 | 97.20 |
| 30 | 97.70 |
| 45 | 98.30 |
| 60 | 98.30 |

### EXAMPLE 2

### Ursodiol Capsule Formulation

| Sr. No. | Ingredients | Mg/Capsule | % W/W |
|---|---|---|---|
| 1. | Ursodiol (ratio of micronized and | 300 | 85.71 |
| | unmicronized particle size is 90:10) | | |
| 2. | Starch | 46.5 | 13.29 |
| 3. | Aerosil | 1.75 | 0.50 |
| 4. | Magnesium Stearate | 1.75 | 0.50 |
| | Total | 350 | 100.00 |

### Procedure

1. Ingredients 1, 2, and 3 were geometrically blended.
2. These ingredients were transferred in blender and mixed for 10 minutes.
3. Ingredient 4 was transferred in blender and mixed for 3 minutes.
4. The final blend is filled into capsules.

1. A pharmaceutical composition comprising a therapeutically effective amount of active pharmaceutical ingredient that is Ursodiol or a pharmaceutically acceptable salt thereof with one or more pharmaceutically acceptable excipients, wherein said active pharmaceutical ingredient is present in the form of micronized and unmicronized particles in a ratio of about 5:95 to about 95:5.

2. The pharmaceutical composition of claim 1, wherein said micronized particles have an average particle size less than about 20 microns.

3. The pharmaceutical composition of claim 1, wherein said micronized particles have an average particle size less than about 10 microns.

4. The pharmaceutical composition of claim 1, wherein said micronized particles have an average particle size less than about 7 microns.

5. The pharmaceutical composition of claim 1, wherein said unmicronized particles have an average particle size not more than about 350 microns.

6. The pharmaceutical composition of claim 1, wherein said unmicronized particles have an average particle size not less than about 150 microns.

7. The pharmaceutical composition of claim 1, wherein the composition is in the form of tablet, capsule, a powder suspension in a liquid or aerosol.

8. The pharmaceutical composition of claim 1, wherein said one or more excipients is selected from disintegrants, diluents, binder, flavoring agents, lubricants, fillers, glidents, sweetening agents, coloring agents, stabilizers, masking agents, wetting agents, and suspending agents.

9. A process for preparing a pharmaceutical composition according to claim 1, which comprises micronizing Ursodiol to obtain micronized ursodiol having average particle size less than 20 micron, mixing micronzed ursodilol with unmicronized ursodiol and one or more pharmaceutically acceptable excipients.

10. (canceled)
